(19) United States
(12) Patent Application Publication
(10) Pub. No.: US 2024/0408007 A1
(43) Pub. Date: Dec. 12, 2024

(54) HYDROCOLLOID BLEND COMPOSITIONS FOR TOPICAL APPLICATION

(71) Applicants: NUTRITION & BIOSCIENCES USA 1, LLC, ROCHESTER, NY (US); DANISCO INDIA PVT. LTD., NEW DEHLI (IN)

(72) Inventors: ATUL ASHOK KUMAR LOHADE, NEW DELHI (IN); VINAY MULEY, NEW DELHI (IN); SHRIKANT KHOT, ROCHESTER, NY (US)

(21) Appl. No.: 18/697,610

(22) PCT Filed: Sep. 30, 2022
(86) PCT No.: PCT/EP2022/077308

§ 371 Date: Apr. 1, 2024

(30) Foreign Application Priority Data
Oct. 1, 2021 (IN) 202111044611
Dec. 15, 2021 (EP) 21214866.2

(51) Int. Cl. A61K 9/06; A61K 9/00; A61K 47/34; A61K 47/36; A61K 47/38

(52) U.S. Cl. CPC A61K 9/06; A61K 9/0014; A61K 47/34; A61K 47/36; A61K 47/38

(57) ABSTRACT

This specification relates to a composition for topical application on the body surface, such as the skin or mucous membranes. Accordingly, this specification relates to pharmaceutically acceptable blends suitable for preparing pharmaceutical formulations. Furthermore, this specification relates to methods for preparing both blends and pharmaceutical formulations, and their use for pharmaceutical administration.

### FIELD

This specification relates to a composition for topical application on the body surface, such as the skin or mucous membranes. Accordingly, this specification relates to pharmaceutically acceptable blends suitable for preparing pharmaceutical formulations. Furthermore, this specification relates to methods for preparing both blends and pharmaceutical formulations, and their use for pharmaceutical/cosmeceutical administration.

### BACKGROUND

Delivery of active agents across the skin or mucous membranes is convenient, pain-free, and non-invasive. Such transdermal or topical drug delivery is typically restricted to low molecular weight drugs and drugs with specific lipophilic/hydrophilic balance able to penetrate the stratum corneum due to low diffusion rate of many pharmaceutical compounds.

Effective transdermal drug delivery is usually limited by effective permeation kinetics, the length of time needed for permeation, the need for a frequent dosing regimen, and the volume size of a transdermal composition needed to deliver a sufficient therapeutic amount of the active agent through the skin or mucous membrane.

An example of a well-known transdermal drug is diclofenac (2-(2,6-dichloranilino) phenylacetic acid) and its formulation as Voltaren® Gel 1% which comprises 1% diclofenac sodium. Voltaren® is indicated in the USA for the relief of the pain due to osteoarthritis of joints amenable to topical treatment. U.S. Pat. No. 9,468,618 B2 disclose one approach of formulating 10% diclofenac sodium gel for once a day formulation to provide pain relief to the patients. U.S. Pat. No. 8,716,340 B2 disclose a diclofenac sodium gel formulation formulated using Carbomer as a gelling agent. U.S. Pat. No. 10,117,829 B2 disclose a transdermal or transmucosal formulation comprising 3% wt of diclofenac, 45% wt of ethanol, 20% wt of propylene glycol, 5% wt of diethylene glycol monoethyl ether, and 3% wt of myristyl alcohol in the form of a gel.

A large number of commercially available topical formulations showed the requirement of increased time for permeation, increased dosing frequency, excess quantity to achieve required therapeutic action, necessary physical attribute of the formulation like skin feel and rheology. To improve these aspects, special ingredients may be added to the composition which are non-compatible with other ingredient such as organosilicones addition in water-based system,

In addition, manufacture steps for the formulation require multiple steps, different temperature conditions like high temperature to melt the solids/disperse the excipient, requirement of adjustment of pH to prepare gel/cream formulation. This makes manufacturing process cumbersome and time consuming. Overall, this leads to increased cost of the formulation. This represents the need of enhanced therapeutic performance of the topical systems, better skin-feel and rheology, simplified manufacture process and stable aqueous organosilicons formulation. Thus, there seem to be a need for improved pharmaceutically acceptable blends, which blends may be used in pharmaceutical formulations suitable for topical use.

### BRIEF DESCRIPTION OF THE DRAWINGS

FIG. 1. Comparative diffusion evaluation of benchmark formulation and different Silicone Hydrocolloid blend-based diclofenac formulation {Silicone Methocel blend (Si-Me), Silicone Sodium alginate blend (Si-Sod Alg), Silicone-Carrageenan blend (Si-CGN), Silicone Xanthan gum blend (Si XG)}

### SUMMARY

It is an object of embodiments of the specification to provide pharmaceutically acceptable blends, which blends may be used in pharmaceutical and/or cosmeceutical formulations suitable for topical use, i.e. in topical drug delivery systems.

This specification relates in a broad aspect to blends comprising an organopolysiloxane thickener and a hydrocolloid, which blends may be used to prepare formulations suitable for topical use, such as in gels, ointments, cream, lotions and the like.

Accordingly, in a first aspect, this specification relates to a pharmaceutically acceptable blend comprising i) 1 to 30% (w/w) of an organopolysiloxane thickener; ii) 0.5 to 8% of a hydrocolloid; iii) 0.5 to 5% (w/w) of a first emulsifier; and water of not less than 40%, and optionally one or more pharmaceutically acceptable preservative, solvent in an amount of not more than 30%, and/or a pH controlling agent.

It is to be understood that in this first aspect of the invention, the organopolysiloxane thickener and hydrocolloid are mixed to become a blend in the presence of water and optionally preservatives and other solvents, pH controlling agents and the like, however which blend is without any active pharmaceutical ingredient. This blend may then be used in many different pharmaceutical systems of gels, creams, lotions, wherein one of more active pharmaceutical ingredient is present to provide a formulation with increased diffusion rate of the active ingredient at the site of application, such as on the skin.

It is also to be understood that the term “first emulsifier” merely refers to the emulsifier used for the blend without the API. The “first emulsifier” and the “second emulsifier” used in the formulation including the API may be the same specific emulsifier compound but may also be different compounds.

Accordingly, in another aspect, this specification relates to a pharmaceutically acceptable formulation, such as a gel suitable for topical use comprising; i) a pharmaceutically acceptable blend according to this specification in an amount of 40-80% (w/w); ii) 1 to 3% (w/w) of at least one active pharmaceutical ingredient (API); iv) 0.5 to 5% (w/w) of a second emulsifier; and optionally one or more pharmaceutically acceptable additional preservative, solvent in an amount of not more than 30%, pH controlling agent, and/or water.

In another aspect, this specification relates to a method of making a pharmaceutically acceptable blend, which method includes the steps of mixing the following ingredients: i) 1 to 30% (w/w) of an organopolysiloxane thickener; ii) 1 to 8% of a hydrocolloid; iii) 0.5 to 5% (w/w) an emulsifier; and water of not less than 40%, and optionally one or more pharmaceutically acceptable preservative, solvent in an amount of not more than 30%, and/or a pH controlling agent to make a pharmaceutically acceptable blend.

In another aspect, this specification relates to the use of a pharmaceutically acceptable blend according to this specification for preparing a pharmaceutical administration, such as a gel or a lotion cream.

In another aspect, this specification relates to the use of a pharmaceutical formulation according to this specification for topical pharmaceutical administration.

### DETAILED DESCRIPTION

As discussed above, this specification relates to topical drug delivery systems. Many advantages are envisioned for these systems.
Drug delivery efficiency: Increased flux helps enhanced therapeutic efficacy of the delivery system. This specification provides for drug delivery systems with increased diffusion rate of the active ingredient. This obviously has many advantages of being able to offer systems with more effective dosage effects, such as by increased or faster diffusion of an active pharmaceutical ingredient.
This specification further provides for application in different delivery platforms: The blend offers advantage to formulate different topical formulations viz., Lotion, gel, cream etc. Consumer wants a choice of formulation rheology; the blend provides the flexibility of the same.
It is expected that embodiments in this specification may reduce process time, cost and increased process efficiency of the formulations described herein: The blend formulation provides ready emulsion of blend. This makes one step process of mixing other ingredients to formulate finished product. This avoids multiple step operation and thereby increased process efficiency and reduce cost.
It is also expected that embodiments in this specification may reduce the costs of the final topical formulations: Silicones offer good skin feel, but formulations with only silicone are costly. Therefore, a need exists to reduce formulation costs when using silicone. One way to reduce the costs of silicone formulations is to incorporate water into the formulation.

The pharmaceutically acceptable blends and formulations described herein comprises several different ingredients as described in more details in the following:

As used herein the terms “organopolysiloxane thickener” or “polysiloxane” or “silicone” refers to a polymer made up of siloxane. In some embodiments, the organopolysiloxane thickener is a non-polar volatile siloxane, such as methylsiloxane or a polydimethylsiloxane. According to some embodiments, the polydimethylsiloxane is a linear polydimethylsiloxane or a cyclic polydimethylsiloxane. According to further embodiments, the polydimethylsiloxane is selected from the group consisting of hexamethyldisiloxane, heptamethyloctyltrisiloxane octamethylcyclotetrasiloxane, octamethyltrisiloxane, decamethylcyclopentasiloxane, decamethyltetrasiloxane, dodecamethylpentasiloxane, dodecamethylcyclohexasiloxane, and a combination thereof. According to a certain embodiment, the polydimethylsiloxane is hexamethyldisiloxane.

In some specific embodiments, the organopolysiloxane thickener is selected from hexamethyldisiloxane, cyclopentasiloxane, cyclohexaxiloxane, alkylmethyl silicone polyglycol, dimethiconol, diphenylsiloxane-dimethylsiloxane copolymers, polyphenylmethylsiloxane, vinylphenylsiloxane-phenylmethylsiloxane copolymer, trifluoropropylmethylsiloxane-dimethylsiloxane copolymer, diethylsiloxane-dimethylsiloxane copolymer, vinylmethylsiloxane-dimethylsiloxane copolymer, vinylmethylsiloxane homopolymers, polyphenyl-(dimethylhydrosiloxy) siloxane, methylhydrosiloxane-phenylmethylsiloxane copolymer, methylhydrosiloxane-dimethylsiloxane copolymers, polymethylhydrosiloxanes, polyethylhydrosiloxane, triethylsiloxane, methylhydrosiloxane-phenyloctylmethylsiloxane copolymer, methylhydrosiloxane-phenyloctylmethylsiloxane terpolymer, or combinations thereof. In some embodiments a polydimethylsiloxane is dimethylhydroxysiloxy-terminated, or trimethylsiloxy-terminated.

### Hydrocolloids

The term “hydrocolloid” as used herein refers to a substance usually a polysaccharide that is colloidally dispersible in water changing the rheology of water by raising the viscosity and/or inducing gelation of the substance in which it is used. Suitable hydrocolloids include cellulose such as methylcellulose, hydroxypropyl methylcellulose, sodium carboxymethylcellulose, alginates, such as alginate salts, carob gum, guar gum, xanthan gum, konjac gum, locust bean gum, gellan gum, such as LA-gellan gum, and curdlan gum pectin, such as LM pectin, and agar. In some embodiments, a hydrocolloid is an alginate, such as sodium alginate. In some embodiments, a hydrocolloid is a seaweed like carrageenan. Other suitable hydrocolloids are nonionic poly (ethylene oxide) polymers available in a wide variety of viscosity grades corresponding to molecular weight, ranging from approximate 100,000 to 7,000,000 Da. POLYOX™ is available from IFF, Nutrition & Biosciences.

As discussed herein, the pharmaceutical blends and compositions of this specification may contain alginate. The alginate may be provided in any suitable form. For example, the alginate will typically be provided in the form of a salt. In one aspect the alginate is selected from sodium alginate, potassium alginate and mixtures thereof. In one aspect the alginate is or comprises sodium alginate. In one aspect the alginate is or comprises potassium alginate. In one aspect the alginate is or comprises a mixture of sodium alginate and potassium alginate. In one aspect the alginate is sodium alginate. In one aspect the alginate is potassium alginate. On one aspect the alginate is a mixture of sodium alginate and potassium alginate.

In some embodiments is used a low molecular weight alginate, which refer to depolymerized alginic acid material with a weight average molecular weight below of 350,000 Da, such as a weight average molecular weight in the range of 300,000-2,000 Da.

The molecular weight of the alginate is not particularly limited. In some embodiments, a weight average molecular weight of 250,000 to 350,000 is used. Also, the degree of esterification is not particularly limited, but may in some embodiments be 20% by weight to 95% by weight, such as 60% by weight to 95% by weight, such as 75% by weight to 95% by weight.

The alginate can generally be derived from natural products or synthesized. Alginates, derived from, inter alia, brown seaweeds are linear, unbranched bio-polymers consisting of (1-4)-linked β-D-mannuronic acid (M) and α-L-guluronic acid (G) residues. Alginates are not random copolymers but consist of blocks of similar and alternating sequences of residues, for example, MMMM, GGGG, and GMGM. In extracted form alginate absorbs water quickly. The physical properties of alginates may depend on the relative proportion of the M and G blocks. Gel formation at neutral pH requires a calcium source to provide calcium ion to interact with G-blocks. The greater the proportion of these G-blocks, the greater the gel strength.

“Alginate” is the term usually used for the salts of alginic acid, but it can also refer to all the derivatives of alginic acid and alginic acid itself; Alginate is present in the cell walls of brown algae (Phaeophyceae sp.) as the calcium, magnesium and sodium salts of alginic acid. Dry, powdered, sodium alginate or potassium alginate may be obtained from an extraction process of this brown algae. The seaweed residue is then removed by filtration and the remaining alginate may then be recovered from the aqueous solution.

Another way to recover the sodium alginate from the initial extraction solution is to add a calcium salt. This causes calcium alginate to form with a fibrous texture; it does not dissolve in water and can be separated from it. The separated calcium alginate is suspended in water and acid is added to convert it into alginic acid. This fibrous alginic acid is easily separated, placed in a planetary type mixer with alcohol, and sodium, potassium or calcium carbonate is gradually added to the paste until all the alginic acid is converted to sodium, potassium or calcium alginate. The paste of sodium, potassium or calcium alginate is sometimes extruded into pellets that are then dried and milled.

Optimal molecular weights and viscosities may vary depending on the specific polymer used for practicing the present invention. For any specific polymer optimal molecular weights and viscosities may be measured and selected by standard methods available for the person skilled in the art.

E.g. for alginates suitable for use in the practice of this invention, this hydrocolloid will typically have a molecular weight such that they exhibit a viscosity in the range of 50-1,000 mPa·s. when measured at 1 weight % at 20° C. using Brookfield type RV (e.g. RVT, RVF, RVTDV) with Brookfield RV using the appropriate spindle for the viscosity range in question. The appropriate spindle for the viscosity determination can be readily determined by one of ordinary skill in the art, based on the equipment model and the viscosity range. In some embodiment, such alginates will exhibit a viscosity of between 200 and 800 when so measured, such as between 400 and 600 mPa·s when so measured. Spindle #2 can be used for viscosity measurements in a desired viscosity range, with the above-specified equipment. In some embodiments, a high G type sodium alginate is used. A high G type sodium alginate means that the alginate(s) employed in the practice of this specification possess an average of at least 50 percent adjacent G units. In some embodiments the alginate will possess an average of at least 52 percent adjacent G units; and in other embodiments such alginate will possess an average of at least 55 percent or more of adjacent G units, as such higher the content of adjacent G units may result in improved product textures.

Examples of commercially available alginate include IFF (formerly DuPont) under the Protanal, Manucol and Manugel brand names, such as Manugel DMB, Manugel GHB, Manugel GMB, Protanal® LFR 5/60, Protanal® CR 8133, Manucol® LKX, Protanal® CR 8223, Aquateric® N100, Pronova® UP LVG, Pronova® UP MVG, PROTANAL 6650, MANUCOL DMF, Manucol DM, PROTANAL SF 120 RB, Protanal® ME6240, Other exemplary commercially available alginates include, but are not limited to GRINDSTED® Alginate FD/PH 120, GRINDSTED® Alginate FD/PH 125, GRINDSTED® Alginate FD/PH 127, GRINDSTED® Alginate FD/PH 150, GRINDSTED® Alginate FD/PH 155, GRINDSTED® Alginate FD/PH 157, GRINDSTED® Alginate FD/PH 170, GRINDSTED® Alginate FD/PH 175, GRINDSTED® Alginate FD/PH 222, GRINDSTED® Alginate FD/PH 275, GRINDSTED® Alginate PH 460, and GRINDSTED® Alginate PH 490.

In addition, in this specification, one type of alginate may be used alone, or two types of alginate may be used in combination.

Another suitable hydrocolloid that may be used in the pharmaceutically acceptable blends or pharmaceutical formulations according to this specification is pectin.

The term “pectin” is to be understood as a water-soluble form of pectic substance obtained by extraction of pectin from a plant material. Pectin has a structure comprising blocks of linear galacturonan chains (polymer of α-(1-4)-linked-D-galacturonic acid) which are interrupted with rhamno-galacturonan backbones (polymers of the repeating disaccharide α-(1-4)-D-galacturonic acid-α-(1-2)-L-rhamnose), which often have side chains of polymeric arabinogalactans glycosidic linked to the O-3 or O-4 positions of L-rhamnose. The galacturonan sequences can have D-xylose and D-apiose glycosidic linked to their O-2 or O-3 positions, which also can be substituted with ester-linked acetyl groups. The long chains of α-(1-4)-linked D-galacturonic acid residues are commonly referred to as “smooth regions”, whereas the highly branched rhamnogalacturonan regions are commonly referred to as the “hairy regions”.

Pectin is a commonly and important polysaccharide with applications in both foods and pharmaceuticals and many commercial sources exist. Most sources of commercial pectin products are citrus peel and apple pomace in which protopectin represents 10-40% by weight of the dry matter.

Pectin is present in almost all higher plants. Some by-products of the food industry are used for pectin extraction, such as citrus peels (by-products of citrus juice production), apple pomace (by-products of apple juice production), beets (by-products of beet sugar industry), slightly extended to Potato fiber, sunflower heads (by-product of oil production) and onions (May 1990, Carbohydr. Polymers, 12:79-99). A typical method for extracting hypermethylated (HM) pectin from pomace or peel is to continue in hot dilute mineral acid at pH 1-3, 50-90° C. for 3-12 hours (Rolin, 2002, in Pectins and their Manipulation; Seymour GB), Knox J P, Blackwell Publishing Ltd, 222-239). The dried citrus peel contains 20-30% pectin (based on dry matter) and the pectin in the dried apple pomace is present in low amounts (10-15%) (Christensen, 1986, Pectins. Food Hydrocolloids, 3, 205-230). Pectin is precipitated by the addition of an alcohol (usually isopropanol but also methanol or ethanol). Finally, the gelatinous material is pressurized, washed, dried and ground (Carbohydr. Polymers, 12:79-99, May 1990). Depending on the process conditions, pectin with a DM of 55-80% was obtained (Rolin, 2002, in Pectins and their Manipulation; Seymour G. B., Knox J. P., Blackwell Publishing Ltd, 222-239).

Hypomethylated (LM) pectin can be obtained by de-esterification of hypermethylated (HM) pectin, primarily by controlling the acidity, temperature and time during the extraction process. In order to produce other types of pectin, the ester may be hydrolyzed as a concentrated liquid or in an alcoholic slurry by acid or base before or during extraction, and then separated and dried. When a base is used, the reaction must be carried out in a low temperature and in an aqueous solution to avoid β-eliminating degradation of the polymer (Kravtchenko et al., 1992, Carbohydrate Polymers, 19, 115-124). LM pectin (e.g., potato pectin) can also be extracted with an aqueous chelating agent such as hexametaphosphate (Voragen et al, 1995, in Food polysaccharides and their applications; Stephen A. M., New York: Marcel Dekker Inc, 287-339). The use of pectin methyl esterase (PME) to produce LM pectin can be used as an alternative to chemical extraction (Christensen, 1986, Pectins. Food Hydrocolloids, 3, 205-230). The conditions and times of the different reactions are different, resulting in pectin with different DE (even DE as low as zero).

Although commercial LM pectin is almost entirely derived from HM pectin, there is a natural source of LM pectin, such as the mature sunflower head (Thakur et al., 1997, Critical Reviews in Food Science and Nutrition, 37 (I): 47-73). One method of producing pectin is described in International Patent Application WO 2013/109721, wherein citrus peel is treated to obtain homogenized citrus peel, the homogenized citrus peel is washed with an organic solvent, followed by a desolventizing and drying step to recover the fiber-containing pectin product or pectin. In some embodiments, a comminuting or pulverizing step is carried out after the drying step.

Alternatively, a suitable pectin product is obtained according to the process described in U.S. Pat. No. 7,833,558, which patent describes a method of providing a fiber-containing pectin product from a plant material which comprises the steps of (i) providing an in situ reaction system by swelling the plant material in an aqueous solution comprising at least one salt, (ii) subjecting pectin present in the swollen plant material from step (i) to a de-esterification treatment, and (iii) separating the de-esterified fiber-containing pectin product. In some embodiments, the plant material is native pectin-containing plant materials including peels or pulp from citrus fruits, such as lemon, orange, mandarin, lime and grapefruit.

Exemplary commercially available pectins include, but are not limited to, apple pectin (SIGMA-ALDRICH, product number 93854), citrus peel pectin (SIGMA-ALDRICH, product number P9135), citrus pectin with a degree of esterification of 60% (SIGMA-ALDRICH, product number P9436) and citrus pectin with a degree of esterification of 90% (SIGMA-ALDRICH, product number P9561), GRINDSTED Pectin RS 400, Andre Pectin AP 101, GENUPECTIN B Rapid Set, UNIPECTIN RS 150, Classic AF 101, GRINDSTED Pectin AMD 780, Andre Pectin AP 140, GENUPECTIN JMJ, UNIPECTIN AYD 20, Classic CM 201/203, GRINDSTED Pectin LC 810, Andre Pectin AP 310, GENUPECTIN LM 18 CG, and UNIPECTIN OF 400, Classic AF 701.

Another suitable hydrocolloid that may be used in the pharmaceutically acceptable blends or pharmaceutical formulations according to this specification is agar. Agar is extracted from red seaweeds (Rhodophyceae) of Gelidiaceae and Gracilariaceae species. Agar contains mainly agarose but also agaropectin, and the former contributes to gelation while the latter weakens the gel. Agar is comprised of the linear polysaccharide polymers based on a disaccharide repeat structure of 3-linked β-D-galactopyranosyl and 4-linked 3,6-anhydro-a-L-galactopyranosyl units (Araki, 1966; Norziah et al., 2006).

Agar can be solubilized at around 90-100° C. and in solution the polymers take the form of random coils. On cooling the liquid agar solution forms a gel by the formation of the double helices. These double helices become linked and form bundles double helices. At junction zones the bundles interact, further forming a three-dimensional network. This network has great ability to immobilize water molecules in its interstices (Norziah et al., 2006). Agar forms brittle gel. Agar has high gelling and melting temperature and high syneresis due to the tight gel network. Agar has optima pH range of 2.5-10.

Other suitable hydrocolloid that may be used in the pharmaceutically acceptable blends or pharmaceutical formulations according to this specification are cellulosic hydrocolloid, such as methyl cellulose, cellulose gum, hydroxypropylmethylcellulose (HPMC), hydroxypropylcellulose (HPC), carboxymethylcellulose (CMC), ethylcellulose (EC), and colloidal microcrystalline cellulose (MCC). One specific methyl cellulose is METHOCEL™, which are a family of water-soluble gums made from natural cellulose (Cellulose structure polysaccharide has same backbone as starch, but-starch glucose units are linked by alpha 1-4 linkages), the most abundant, renewable carbohydrate found in nature. They are inert, high purity powders with no caloric value, and are virtually colorless, odorless, and tasteless. Key Properties of METHOCEL™: Reversible thermal gelation; Water Soluble; Thickening; Emulsification/encapsulation; Film formation; Binding (hot and cold); Air entrainment and foam stability; Freeze thaw stability; Provide soluble fiber. Key Featured Technology for METHOCEL™: Improves extrusion and release properties; Good emulsification & deters separation; Improves Yield.

Exemplary commercially available methyl cellulose products include but are not limited to products of METHOCEL™ grades (IFF), such as METHOCEL™ SG A7C, METHOCEL™ SG A16M, METHOCEL™ MX, Benecel MX, METHOCEL™ Bind 250, METHOCEL™ Bind 092, METHOCEL™ Bind 112 and Metolose MCE 100 TS, High Viscosity METHOCEL™ Premium F4M, K4M, K15M, K100M, A4M, E4M and E10M.

Another specific cellulosic hydrocolloid is carboxymethylcellulose (CMC), such as Sodium carboxymethylcellulose (CMC). Suitable sodium carboxymethylcellulose products may be of the TEXTURECEL™ brand including TEXTURECEL™ PRM 700 PA, TEXTURECEL™ 1,000 PA, TEXTURECEL™ 4,000 PA 07, and TEXTURECEL™™ 10,000 PA, TEXTURECEL™ 1,000 PA 07, TEXTURECEL™ 2,000 PA 07, TEXTURECEL™ PRM 2,400 PA 07, TEXTURECEL™ 20,000 PA 07 or of the AQUALON® or Blanose® brands (Ashland), such as Aqualon® CMC 7H3SXF, Aqualon™ 12M31P, Aqualon™ 12M31XP. Suitable viscosity grades include but are not limited to grades above 10,000, such as up to 20,000. Another suitable hydrocolloid that may be used in the pharmaceutically acceptable blends or pharmaceutical formulations according to this specification is carrageenan.

As will be appreciated by one skilled in the art, ingredients obtained from seaweed of the class of Rhodophyta will contain carrageenan. Carrageenan refers to a family of linear sulfated polysaccharides that are extracted from red edible seaweeds. Carrageenan is a high-molecular-weight polysaccharide made up of repeating galactose units and 3,6 anhydrogalactose (3,6-AG), both sulfated and nonsulfated. The units are joined by alternating α-1,3 and β-1,4 glycosidic linkages. Carrageenan is widely used in the food and other industries as thickening or stabilizing agents. There are three main commercial classes of carrageenan:

These three varieties differ in their degree of sulfation. Kappa carrageenan has one sulfate group per disaccharide, iota carrageenan has two, and lambda carrageenan has three. When used in food products, carrageenan has the EU additive E numbers E407 or E407a when present as “processed eucheuma seaweed”.

Kappa carrageenan forms strong, rigid gels in the presence of potassium ions, and reacts with dairy proteins. It is sourced mainly from Kappaphycus alvarezii. Kappa carrageenan is typically produced by alkaline elimination from mu carrageenan, also isolated Kappaphycus alvarezii. The structures of these two materials and the alkaline conversion is shown below.

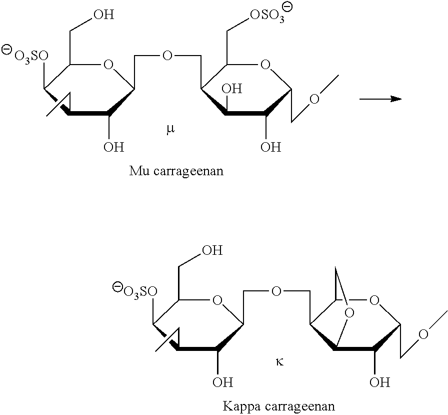

The content of the various forms of carrageenan can be readily determined by one skilled in the art. The examples of the present specification provide a detailed method of how this determination may be made. In one aspect, the content of carrageenan type, such a mu carrageenan is determined in accordance with Determination Process 1 (Determination of Carrageenan types by 1H-NMR).

In one aspect, the in the pharmaceutically acceptable blends or pharmaceutical formulations according to this specification contains mu carrageenan in an amount of at least 0.5 wt. %, such as 1, 2, 3, 4 or 5 wt. % based on the total weight of the pharmaceutically acceptable blend or pharmaceutical formulation.

In one aspect, the pharmaceutically acceptable blends or pharmaceutical formulations according to this specification contain mu carrageenan in an amount of not more than 8 wt. %, such as not more than 7, 6, 5, or 4 wt. %, based on the total weight of the pharmaceutically acceptable blend or pharmaceutical formulation.

Suitable carrageenan products for pharmaceutically acceptable blends or pharmaceutical formulations according to this specification include various commercial carrageenans, such as Avicel PH101, Gelcarin® carrageenan and Viscarin® carrageenan grades, such as Gelcarin® GP-379NF, Viscarin® 101, Viscarin® GP-328NF, Viscarin® GP-209NF, Viscarin® GP109 Gelcarin® GP911, Gelcarin® GP-812NF (IFF Nutrition & Biosciences).

Another suitable hydrocolloid that may be used in the pharmaceutically acceptable blends or pharmaceutical formulations according to this specification is xanthan gum.

Xanthan gum is a long chain polysaccharide composed of glucose, mannose, and glucuronic acid. The backbone of the polysaccharide chain consists of two beta-D-glucose units linked through the 1 and 4 positions. The side chain consists of two mannose and one glucuronic acid, so the chain consists of repeating modules of five sugar units. The side chain is linked to every other glucose of the backbone at the 3 position. About half of the terminal mannose units have a pyruvic acid group linked as a ketal to its 4 and 6 positions. The other mannose unit has an acetyl group at the 6 positions. Two of these chains may be aligned to form a double helix, giving a rather rigid rod configuration that accounts for its high efficiency as a viscosifier of water. The molecular weight of xanthan varies from about one million to 50 million depending upon how it is prepared. Suitable xanthan gum sources are well known to the person skilled in the pharmaceutical and cosmeceutical field and includes xanthan gum provided by Jungbunzlauer or by Monsanto-Kelco Co., or by Rhodia, Inc. Courbevoie, France, Cranbury, NJ USA, such as the Jungbunzlauer XG, KELTROL®, GFS®, KELGUM®, DRICOID®, KELZAN®, KELZANAR, RHODIGEL® RHODIGUM®, RHODICARE® and RHODOPOL® product ranges.

### Active Pharmaceutical Ingredients (API)

It is to be understood that the specific active pharmaceutical ingredients (API) to be used in the formulations of this specification is not essential. Obviously for a specific method of treatment the specific API will also be essential. However, it is envisioned that, in principle, the formulations of this specification may be useful for many different APIs including anti-inflammatory agents, anti-microbial, anti-infective agents, anti-allergic agents, antihistamines, antiproliferative agents, anti-angiogenic agents, anti-oxidants, antihypertensive agents, neuroprotective agents, cell receptor agonists, cell receptor antagonists, immunomodulating agents, immunosuppressive agents, intraocular pressure lowering agents, a2-adrenergic receptor agonists, β-adrenergic receptor antagonists, carbonic anhydrase inhibitors, cholinesterase inhibitor miotics, prostaglandins, prostaglandin receptor agonists, mast cell degranulation inhibitors, thromboxane A2 mimetics, protein kinase inhibitors, prostaglandin F derivatives, prostaglandin F 2a receptor antagonists, retinoids, glucocorticosteroids, UV filters, cyclooxygenase-2 inhibitors, muscarinic agents, steroids, and any combination thereof.

In some embodiments the API is lipophilic. Diclofenac is used in the present disclosure as an example and in one specific embodiment the active pharmaceutical ingredients (API) is Diclofenac.

### Other Excipients

Examples of pH controlling agents include any water-soluble acid such as a carboxylic acid or a mineral acid such as hydrochloric acid, sulphuric acid, and phosphoric acid, monocarboxylic acid such as acetic acid and lactic acid, and polycarboxylic acids such as succinic acid, adipic acid, and citric acid. pH controlling and adjusting agents for pharmaceutical use are well-known for the person skilled in the art.

### Emulsifiers

Emulsifiers for pharmaceutical use are well-known to the person skilled in the art and may comprise one or more compound selected from the group consisting of a sodium stearate, stearic acid, sodium oleate, sodium lauryl sulfate, sodium cetyl sulfate, sulfated castor oil, glycerol monostearate, polyoxyethylene Sorbitan fatty acid ester, such as tween 80, sorbitan fatty acid ester, such as Span 60, polysorbate 60 (sorbitan polyoxyethylene monostearate, polysorbate 40, Tween 40, polyoxyethylene sorbitan monopalmitate) and other sorbitan ester emulsifiers, such as polysorbate 40.

Other suitable emulsifiers may include but not limited to polyoxyethylene hydrogenated castor oil, cetostearyl alcohol, sorbitan monostearate, sorbitan monopalmitate, glyceryl monostearate, sorbitan monolaurate, polyoxyethylene polyoxypropylene block copolymer, medium-chain triglycerides, sucrose fatty acid ester, lecithin or mixtures thereof.

### Pharmaceutically Acceptable Preservatives

Also the use of preservatives in pharmaceutical formulations are well-known to the skilled person in the art. Several preservatives are suitable, and the specific preservative does not generally seem to be essential in the context of this specification. Accordingly the preservative may comprise one or more compounds selected from the list consisting of an aromatic alcohol, such as benzyl alcohol, sodium benzoate, benzoic acid, sorbic acid, benzethonium chloride, benzalkonium chloride, bronopol, methylparaben, ethylparaben, propylparaben, butylparaben, thiomerosal, sodium propionate, chlorhexidine, chlorobutanol, chlorocresol, cresol, imidazolidinyl urea, diazolidinyl urea, phenol, phenylmercuric salts, potassium sorbate, propylene glycol, isopropyl alcohol, or mixtures thereof.

### Solvents

As used herein the solvent refers to any suitable organic solvent able to dissolve the specific API used. Suitable solvents may be selected from the group consisting of glycols, alcohol and fatty acid alcohol and ester and 2-pyrrolidone, such as acetone, acetonitrile, benzyl alcohol, isopropyl alcohol, butyl diglycol, dimethylacetamide, dimethylformamide, dipropylene glycol n-butyl ether, ethylene glycol monoethyl ether, ethylene glycol monomethyl ether, monomethylacetamide, dipropylene glycol monomethyl ether, liquid polyoxyethylene glycols, propylene glycol, 2-pyrrolidone, diethylene glycol monoethyl ether, ethylene glycol, diethyl phthalate fatty acid esters, and a mixture of at least two of these solvents. In some specific embodiments the solvent used is propylene glycol and/or isopropyl alcohol.

### Numbered Embodiments of the Invention

1. A pharmaceutically acceptable blend comprising
i) 1 to 30% (w/w) of an organopolysiloxane thickener;
ii) 0.5 to 8% of a hydrocolloid;
iii) 0.5 to 5% (w/w) of a first emulsifier; and water of not less than 40%, and optionally one or more pharmaceutically acceptable preservative, solvent in an amount of not more than 30%, and/or a pH controlling agent.

2. A pharmaceutically acceptable formulation, such as a gel suitable for topical use comprising;
i) a pharmaceutically acceptable blend as defined in embodiment 1 in an amount of 40-80% (w/w);
ii) 1 to 3% (w/w) of at least one active pharmaceutical ingredient (API);
iv) 0.5 to 5% (w/w) of a second emulsifier; and optionally one or more pharmaceutically acceptable additional preservative, solvent in an amount of not more than 30%, pH controlling agent, and/or water.

3. The pharmaceutically acceptable blend or pharmaceutical formulation according to embodiments 1 or 2, wherein said organopolysiloxane thickener is selected from the list consisting of linear or cyclic polydimethylsiloxane, such as hexamethyldisiloxane, cyclopentasiloxane, cyclohexaxiloxane, alkylmethyl silicone polyglycol, dimethiconol, diphenylsiloxane-dimethylsiloxane copolymers, polyphenylmethylsiloxane, vinylphenylsiloxane-phenylmethylsiloxane copolymer, trifluoropropylmethylsiloxane-dimethylsiloxane copolymer, diethylsiloxane-dimethylsiloxane copolymer, vinylmethylsiloxane-dimethylsiloxane copolymer, vinylmethylsiloxane homopolymers, polyphenyl-(dimethylhydrosiloxy) siloxane, methylhydrosiloxane-phenylmethylsiloxane copolymer, methylhydrosiloxane-dimethylsiloxane copolymers, polymethylhydrosiloxanes, polyethylhydrosiloxane, triethylsiloxane, methylhydrosiloxane-phenyloctylmethylsiloxane copolymer, methylhydrosiloxane-phenyloctylmethylsiloxane terpolymer, or combinations thereof.

4. The pharmaceutically acceptable blend or pharmaceutical formulation according to embodiments 1-3, which organopolysiloxane thickener has a viscosity of 8-30.000 cSt, such as a viscosity greater than about 10,000 cSt, greater than about 12,000 cSt, greater than about 15,000 cSt, greater than about 20,000 cSt, greater than about 30,000 cSt, greater than about 40,000 cSt, greater than about 60,000 cSt, greater than about 80,000 cSt, greater than about 100,000 cSt, greater than about 125,000 cSt, or greater than about 150,000 cSt at about 25° C.

5. The pharmaceutically acceptable blend or pharmaceutical formulation according to any one of embodiments 1-4, which organopolysiloxane thickener has a viscosity of less than about 2,000,000 cSt, less than about 1,000,000 cSt, less than about 500,000 cSt, less than about 450,000 cSt, less than about 400,000 cSt, less than about 350,000 cSt, less than about 300,000 cSt, less than about 250,000 cSt, less than about 200,000 cSt, less than about 100,000 cSt, less than about 50,000 cSt, less than about 40,000 cSt, less than about 30,000 cSt, less than about 20,000 cSt, or less than about 15,000 cSt at about 25° C.

6. The pharmaceutically acceptable blend or pharmaceutical formulation according to any one of embodiments 1-5, wherein said hydrocolloid is a non-cellulosic hydrocolloid selected from the list carrageenan, pectin, alginate, such as sodium alginate and propylene glycol alginate, xanthan gum, guar gum, gum arabic, locust bean gum.

7. The pharmaceutically acceptable blend or pharmaceutical formulation according to any one of embodiments 1-6, wherein said hydrocolloid is an alginate salt, such as a sodium alginate, such as Protanal® CR 8133, Manucol® LKX, Protanal® CR 8223, a propylene glycol alginate, such as Kelcoloid K 3B426 and Protanal Ester SD.

8. The pharmaceutically acceptable blend or pharmaceutical formulation according to embodiment 7, wherein the non-cellulosic hydrocolloid, such as an alginate is present in said pharmaceutically acceptable blend in an amount of from 1% (w/w) to about 5% (w/w), such as from 2% (w/w) to about 4% (w/w), such as from 2% (w/w) to about 3% (w/w).

9. The pharmaceutically acceptable blend or pharmaceutical formulation according to any of embodiments 7 or 8, wherein said alginate has a viscosity of in the range of 50-2,000 mPa·s, such as in the range of 100-1500 mPa·s, such as in the range of 100-1000 mPa·s, such as in the range of 100-900 mPa·s, such as in the range of 200-900 mPa·s, such as in the range of 300-900 mPa·s, such as in the range of 400-900 mPa·s, such as in the range of 500-900 mPa·s, such as in the range of 600-900 mPa·s, when measured at 1.25 weight % at 20° C. using Brookfield type RV with Brookfield RV using the appropriate spindle for the viscosity range in question.

10. The pharmaceutically acceptable blend or pharmaceutical formulation according to any one of embodiments 1-5, wherein said hydrocolloid is a cellulosic hydrocolloid selected from the list cellulose gum, hydroxypropylmethylcellulose (HPMC), hydroxypropylcellulose (HPC), carboxymethylcellulose (CMC), ethylcellulose (EC), methylcellulose (MC), and colloidal microcrystalline cellulose (MCC).

11. The pharmaceutically acceptable blend or pharmaceutical formulation according to embodiment 10, wherein said cellulosic hydrocolloid is selected from methylcellulose (MC), such as METHOCEL A (International Flavors & Fragrances, Inc.), hydroxypropyl methylcellulose (HPMC) with a degree of methoxyl substitution (DS) and of hydroxypropoxyl substitution (MS) corresponding METHOCEL E, K and F (International Flavors & Fragrances, Inc.).

12. The pharmaceutically acceptable blend or pharmaceutical formulation according to embodiment 10 or 11, wherein said cellulosic hydrocolloid has a viscosity of in the range of from 4000 to 120,000 cSt.

13. The pharmaceutically acceptable blend or pharmaceutical formulation according to any one of embodiments 10-12, wherein the cellulosic hydrocolloid is present in said pharmaceutically acceptable blend in an amount of from 0.5% (w/w) to about 5% (w/w), such as from 1% (w/w) to about 5% (w/w), such as from 2% (w/w) to about 4% (w/w), such as from 2% (w/w) to about 3% (w/w).

14. The pharmaceutically acceptable blend or pharmaceutical formulation according to any one of embodiments 1-13, wherein said first and/or second emulsifier comprises one or more compound selected from the group consisting of a sodium stearate, stearic acid, sodium oleate, sodium lauryl sulfate, sodium cetyl sulfate, sulfated castor oil, glycerol monostearate, polyoxyethylene Sorbitan fatty acid ester, such as tween 80, sorbitan fatty acid ester, such as Span 60, polysorbate 60 and other sorbitan ester emulsifiers.

15. The pharmaceutically acceptable blend or pharmaceutical formulation according to any one of embodiments 1-14, wherein said preservative comprises one or more compounds selected from the list consisting of an aromatic alcohol, such as benzyl alcohol, sodium benzoate, benzoic acid, sorbic acid, benzethonium chloride, benzalkonium chloride, bronopol, methylparaben, ethylparaben, propylparaben, butylparaben, thiomerosal, sodium propionate, chlorhexidine, chlorobutanol, chlorocresol, cresol, imidazolidinyl urea, diazolidinyl urea, phenol, phenylmercuric salts, potassium sorbate, propylene glycol, isopropyl alcohol, or mixtures thereof.

16. The pharmaceutically acceptable blend or pharmaceutical formulation according to any one of embodiments 1-15, wherein said solvent is selected from the group consisting of glycols, alcohol and fatty acid alcohol and ester and 2-pyrrolidone, such as acetone, acetonitrile, benzyl alcohol, isopropyl alcohol, butyl diglycol, dimethylacetamide, dimethylformamide, dipropylene glycol n-butyl ether, ethylene glycol monoethyl ether, ethylene glycol monomethyl ether, monomethylacetamide, dipropylene glycol monomethyl ether, liquid polyoxyethylene glycols, propylene glycol, 2-pyrrolidone, diethylene glycol monoethyl ether, ethylene glycol, diethyl phthalate fatty acid esters, and a mixture of at least two of these solvents.

17. The pharmaceutically pharmaceutical formulation according to any one of embodiments 1-16, which formulation has a diffusion rate of the active pharmaceutical ingredient (API) in a topical gel of more than 500 ug/cm2/min after 240 min as measured in a diffusion assay using a cellulosic membrane as described herein.

18. The pharmaceutically pharmaceutical formulation according to any one of embodiments 1-17, which active pharmaceutical ingredient (API) is selected from the list consisting of wherein the at least one API is selected from the group consisting of 8-methoxysoralen, acyclovir, aflibercept, an amino acid/urea, aminolevulinic acid, ammonium lactate, anthralin, antipyrine/benzocaine, antipyrine/benzocaine/polycosanol, antyprine bonzocainc/zinc acetate, Arnica montana, balsam peru/castor oil/trypsin, benzalkonium hydrochloride, benzocaine, benzoyl peroxide, betamethasone, betamethasone/clotrimazole, Boswellia, bromelain, bromfenac, calcipotriene, Calendula officinalis, camphor/menthol, capsaicin, carbamide peroxide, chlorhexidine, chloroxylenol/hydrocortisone/pramoxine, ciclopirox, ciprofloxacin, cipro floxacin/dexamethasone, ciprofloxacin/hydrocortisone, Colchicinum autumnale, collagenase, conjugated estrogens, crotamiton, cyclopentolate, cyclosporine, cysteamine, dapiprazole, dexamethasone, dexamethasone/tobramycin, diclofenac, diphenhydramine, diphenhydramine/hydrocortisone, docusate, doxepin, erythromycin, estradiol, Evening Primrose Oil, fluocinolone, fluocinolone/hydrocortisone/tretinoin, fluocinolone/neomycin, fluorescein, fluorometholone, fluorouracil, fluticasone, ganciclovir, gentamicin/prednisolone, ginger, homatropine, hydrocortisone/pramoxine, hydroquinone, ibuprofen, imiquimod, indocyanine green, ivermectin, ketoconazole, ketorolac, ketorolac/phenylephrine, latanoprost, Ledum palustre (marsh-tea), levofloxacin, lidocaine, lidocaine hydrochloride, m-cresyl acetate, mechlorethamine, mepyramine, methyl salicylate, metronidazole, miconazole, minocycline, minoxidil, mometasone, monobenzone, moxifioxacin, naphazoline, neomycin/polymyxin B/hydrocortisone, nystatin, nystatin/triamcinolone, ocriplasmin, ofloxacin, oxymetazoline, papain/urea, pegaptanib, penciclovir, permethrin, phenylephrine, podofilox, podophyllum resin, polymyxin B, pramoxine, prednisolone, prednisolone/sulfacetamide sodium, prilocaine, proparacaine, quercetin, ranibizumab, Rhus toxicodendron (poison ivy), rutin, salicylic acid, sulfacetamide sodium, Symphytum officinale (comfrey), tavaborole, tazarotene, Tea Tree Oil, tetracaine, tetrahydrozoline, timolol, tolnaftate, travoprost, tretinoin, triamcinolone, triclosan, trimethylpsoralen, trolamine salicylate, tropicamide, trypan blue, turmeric, undecylenic acid, urea, vitamin A, vitamin D and vitamin E.

19. The pharmaceutically pharmaceutical formulation according to any one of embodiments 1-18, comprising water present in an amount of not less than 45%, such as not less than 50%, such as not less than 55%, such as not less than 60%, such as not less than 65%, such as not less than 70%, such as in the range of 70-85%.

20. Method of making a pharmaceutically acceptable blend, which method includes the steps of mixing the following ingredients:
i) 1 to 30% (w/w) of an organopolysiloxane thickener;
ii) 1 to 8% of a hydrocolloid;
iii) 0.5 to 5% (w/w) an emulsifier; and water of not less than 40%, and optionally one or more pharmaceutically acceptable preservative, solvent in an amount of not more than 30%, and/or a pH controlling agent to make a pharmaceutically acceptable blend.

21. The method of embodiment 20, which includes the steps of
i) dispersing the hydrocolloid in the water at room temperature or optionally at a temperature of between 50-90° C., such as between 60-90° C., such as 70-90° C., such as for a period of at least 10, such as 20 or 30 min;
ii) mixing the organopolysiloxane thickener and the dispersed hydrocolloid at room temperature or optionally at a temperature of between 40-80° C., such as between 50-70° C., such as 50-60° C.

22. Method of making a pharmaceutical formulation, such as a gel suitable for topical use, which method includes the steps of mixing the following ingredients:
i) a pharmaceutically acceptable blend as prepared according to the method of embodiments 20 or 21 in an amount of 40-80% (w/w);
ii) 1 to 3% (w/w) of at least one active pharmaceutical ingredient (API);
iii) 0.5 to 5% (w/w) a second emulsifier; and optionally one or more pharmaceutically acceptable additional preservative, solvent, a pH controlling agent and/or water, to make a pharmaceutically acceptable formulation, such as a topical gel.

23. The method according to any one of embodiments 16-18, which pharmaceutically acceptable blend or pharmaceutical formulation is as defined in any one of embodiments 1-19.

24. Use of pharmaceutically acceptable blend as defined in any one of embodiments 1-19 for preparing a pharmaceutical administration, such as a gel, ointment, cream, or lotion cream.

25. Use of a pharmaceutical formulation, such as a gel, ointment, cream, or lotion, as defined in any one of embodiments 1-19 for topical pharmaceutical administration.

### EXPERIMENTAL SECTION

### Examples 1

### Equipment Used to Manufacture the Formulation

1. Heater
2. Overhead stirrer
3. Weighing balance

### Blend Preparation:

### Hydrocolloid and Silicone Blend

### Composition (Batch Size 100 gm):

| | | Hypromellose | Carra- | Sodium | |
|---|---|---|---|---|---|
| | | (Methocel | geenan | Alginate | Xanthan |
| Sr. | | K100M | (Viscarin | (Protanal | Gum |
| No | Ingredients | Premium) | 101) | CR 8223) | (XG 80) |

Organic/ Oil Phase

| | | | | | |
|---|---|---|---|---|---|
| 1 | Silicone Fluid DC | 15 | 15 | 20 | 15 |
| | 9120 (12500 cST) | | | | |
| 2 | Tween 80 | 1.5 | 1.5 | 1.5 | 1.5 |

Aqueous phase

| | | | | | |
|---|---|---|---|---|---|
| 3 | Hydrocolloids* | 2.1 | 4.0 | 2.8 | 2.6 |
| 4 | Purified water | 80.4 | 78.5 | 74.7 | 79.9 |
| 5 | Benzyl alcohol | 1.0 | 1.0 | 1.0 | 1.0 |

*{Hydrocolloids: Hypromellose, Carrageenan, Sodium Alginate, Xanthan gum}, All quantities measured in gm

### Process:

### I) Preparation of Organic/Oil Phase:

1. Take Silicone fluid DC 9120 in a container, add Tween 80 in this container mix it for 15 min.

### II) Preparation of Aqueous Phase:

1. For Hypromellose: Heat the purified water at 70 to 80° C. Add and disperse Methocel K100M premium into hot water and disperse well.
   For Carrageenan: Heat the purified water at 70 to 80° C. Add and dissolve Carrageenan into hot water and stir the mixture for 30 min.
   For Sodium alginate and Xanthan gum: Add and dissolve in purified water.

### III) Preparation of Emulsion and Gel:

2. Add hot/as is dispersion to step 1 under stirring to form uniform gel.
3. Stir the mixture for another 45 minutes
4. Add Benzyl alcohol into the preparation.
5. Stir the mixture for another 15 minutes.

### Preparation of Diclofenac Gel

### Composition (Batch Size 100 gm):

| | | Si- | Si- | Si- | Si- |
|---|---|---|---|---|---|
| | | Hypro- | Carra- | Sodium | Xanthan |
| Sr. | | mellose | geenan | Alginate | Gum |
| No | Ingredients | Blend | Blend | Blend | Blend |
| 1 | Diclofenac | 1.0 | 1.0 | 1.0 | 1.0 |
| | Sodium | | | | |
| 2 | Propylene glycol | 20.0 | 15.0 | 15.0 | 15.0 |
| 3 | Isopropyl alcohol | 10.0 | 10.0 | 10.0 | 10.0 |
| 4 | Tween 80 | 1.0 | 1.0 | 1.0 | 1.0 |
| 5 | Silicone- | 55.0 | 70.0 | 60.0 | 60.0 |
| | Hydrocolloid | | | | |
| | Blend* | | | | |
| 6 | Purified water | Quantity | Quantity | Quantity | Quantity |
| | | sufficient | sufficient | sufficient | sufficient |
| | | to 100 gm | to 100 gm | to 100 gm | to 100 gm |
| 7 | Benzyl alcohol | 1.0 | 1.0 | 1.0 | 1.0 |
| 8 | pH modifier** | — | — | — | — |

*Silicone-Hydrocolloid blend—Si—Me/Si-Sod Alg/Si-CGN/Si-XG),
**pH modifier (0.1 N Sodium hydroxide or citric acid) is used in very small quantity to adjust pH 7.4
All quantities measured in gm

### Process

### I) Preparation

1. Add and dissolve Diclofenac sodium in propylene glycol.
2. Add Isopropyl alcohol and Tween 80 to step 1, and stir for 10 min.
3. Add hydrocolloid/Silicone blend to the step 2 add required quantity of purified water and mix it for 30 min
4. Add Benzyl alcohol into the preparation. Stir the mixture for another 15 minutes.
5. Adjust pH using pH modifier

### Diffusion Study

Prior to study, cellulose nitrate membrane was soaked in pH 7.4 phosphate buffer for 30 min.

The membrane was mounted between the donor and receptor compartments.

Phosphate buffer (pH 7.4) was added into the receptor compartment (12 ml) and maintained at 32±0.5° C. under stirring at 400 rpm

The gel formulations of the invention were applied on the cellulose nitrate membrane and the compartments were clamped together.

Aliquots (0.5 mL) were withdrawn at defined time interval and volume withdrawn was replenished with an equal quantity of buffer solution at each time point. The samples were analyzed using HPLC.

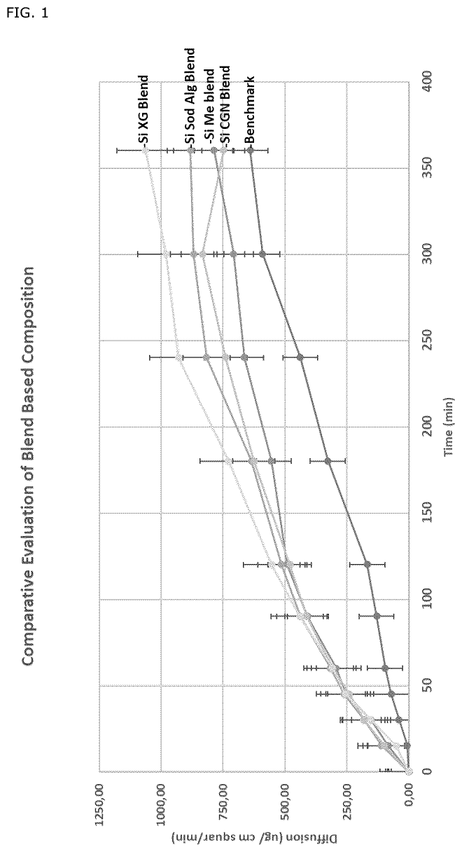

Comparative diffusion study of the formulations of the invention with commercial benchmark (available in India) showed enhanced flux. (see FIG. 1).

1. A pharmaceutically acceptable blend comprising:
i) 1 to 30% (w/w) of an organopolysiloxane thickener,
ii) 0.5 to 8% of a hydrocolloid,
iii) 0.5 to 5% (w/w) of a first emulsifier, and
iv) water of not less than 40%.

2. A pharmaceutically acceptable formulation comprising;
i) a pharmaceutically acceptable blend as defined in claim 1 in an amount of 40-80% (w/w),
ii) 1 to 3% (w/w) of at least one active pharmaceutical ingredient (API), and
iii) 0.5 to 5% (w/w) of a second emulsifier.

3. The pharmaceutically acceptable blend according to claim 1, wherein said organopolysiloxane thickener is selected from hexamethyldisiloxane, cyclopentasiloxane, cyclohexaxiloxane, alkylmethyl silicone polyglycol, dimethiconol, diphenylsiloxane-dimethylsiloxane copolymers, polyphenylmethylsiloxane, vinylphenylsiloxane-phenylmethylsiloxane copolymer, trifluoropropylmethylsiloxane-dimethylsiloxane copolymer, diethylsiloxane-dimethylsiloxane copolymer, vinylmethylsiloxane-dimethylsiloxane copolymer, vinylmethylsiloxane homopolymers, polyphenyl-(dimethylhydrosiloxy) siloxane, methylhydrosiloxane-phenylmethylsiloxane copolymer, methylhydrosiloxane-dimethylsiloxane copolymers, polymethylhydrosiloxanes, polyethylhydrosiloxane, triethylsiloxane, methylhydrosiloxane-phenyloctylmethylsiloxane copolymer, methylhydrosiloxane-phenyloctylmethylsiloxane terpolymer, and combinations thereof.

4. The pharmaceutically acceptable blend according to claim 1 wherein the organopolysiloxane thickener has a viscosity of less than about 2,000,000 cSt at 25° C.

5. The pharmaceutically acceptable blend according to claim 1, wherein said hydrocolloid is a non-cellulosic hydrocolloid selected from carrageenan, pectin, alginate, xanthan gum, guar gum, gum arabic, locust bean gum, and a propylene glycol alginate.

6. The pharmaceutically acceptable blend or pharmaceutical formulation according to claim 1 wherein said hydrocolloid is a cellulosic hydrocolloid selected from cellulose gum, hydroxypropylmethylcellulose (HPMC), hydroxypropylcellulose (HPC), carboxymethylcellulose (CMC), ethylcellulose (EC), methylcellulose (MC), and colloidal microcrystalline cellulose (MCC).

7. The pharmaceutically acceptable blend according to claim 6, wherein said cellulosic hydrocolloid is selected from methylcellulose (MC), and hydroxypropyl methylcellulose (HPMC) with a degree of methoxyl substitution (DS) and of hydroxypropoxyl substitution (MS) corresponding METHOCEL E, K and F (International Flavors & Fragrances, Inc.).

8. The pharmaceutically acceptable blend according to claim 6, wherein said cellulosic hydrocolloid has a viscosity of in the range of from 4000 to 120,000 cSt.

9. The pharmaceutically acceptable blend according to claim 6, wherein the cellulosic hydrocolloid is present in said pharmaceutically acceptable blend in an amount of from 0.5% (w/w) to about 5% (w/w).

10. A method of making a pharmaceutically acceptable blend, which method includes the steps of mixing the following ingredients:
i) 1 to 30% (w/w) of an organopolysiloxane thickener,
ii) 1 to 8% of a hydrocolloid,
iii) 0.5 to 5% (w/w) an emulsifier, and
iv) water of not less than 40%.

11. The method of claim 10, which includes the steps of:
i) dispersing the hydrocolloid in the water at room temperature or at a temperature of between 50-90° C. for at least 10 min, and
ii) mixing the organopolysiloxane thickener and the dispersed hydrocolloid at room temperature or at a temperature of between 40-80° C.

12. A method of making a pharmaceutical formulation, which method includes the steps of mixing the following ingredients:
i) a pharmaceutically acceptable blend as prepared according to the method of 10 in an amount of 40-80% (w/w);
ii) 1 to 3% (w/w) of at least one active pharmaceutical ingredient (API); and
iii) 0.5 to 5% (w/w) a second emulsifier.

13. (canceled)

14. A gel, ointment, cream or lotion, wherein the gel, ointment, cream or lotion comprises the pharmaceutically acceptable blend defined in claim 1.

15. A gel, ointment, cream or lotion suitable for topical pharmaceutical administration, wherein the gel, ointment, cream or lotion comprises the pharmaceutical formulation defined in claim 1.

16. A pharmaceutically acceptable blend according to claim 1, wherein the blend further comprises one or more pharmaceutically acceptable preservative(s), a solvent in an amount of not more than 30%, and/or a pH controlling agent.

17. A pharmaceutically acceptable formulation according to claim 2, wherein the pharmaceutically acceptable formulation is in the form of a gel suitable for topical use.

18. A pharmaceutically acceptable formulation according to claim 2, wherein the pharmaceutically acceptable formulation further comprises one or more pharmaceutically acceptable additional preservative(s), a solvent in an amount of not more than 30%, a pH controlling agent, and/or water.

19. The pharmaceutically acceptable blend according to claim 5, wherein the hydrocolloid comprises an alginate present in the pharmaceutically acceptable blend in an amount of from 1% (w/w) to about 5% (w/w).

20. The method of claim 11, wherein:
i) the hydrocolloid is dispersed in the water at room temperature for at least 10 min, and
ii) the organopolysiloxane thickener is mixed with the dispersed hydrocolloid at room temperature.

21. The method of claim 11, wherein the hydrocolloid is dispersed in the water at a temperature of between 50-90° C. for at least 10 min.

22. The method of claim 11, wherein the organopolysiloxane thickener is mixed with the dispersed hydrocolloid at a temperature of between 40-80° C.
